(19) United States
(12) Patent Application Publication
(10) Pub. No.: US 2005/0186257 A1
(43) Pub. Date: Aug. 25, 2005

(54) Dissolvable film and method of manufacture

(76) Inventors: Todd Manegold, Hoboken, NJ (US); Richard G. Zielinski, East Brunswick, NJ (US)

Correspondence Address: Cynthia L. Foulke;NATIONAL STARCH AND CHEMICAL COMPANY, 10 Finderne Avenue, Bridgewater, NJ, 08807-0500, US

(21) Appl. No.: 10/783,080

(22) Filed: Feb. 20, 2004

(51) Int. Cl. A61K 31/522; A61K 9/70

(52) U.S. Cl. 424/443; 514/263.31

(57) ABSTRACT

Dissolvable films for the administration of an active ingredient having a water solubility of less than about 1 g/4 mL at room temperature.

### FIELD OF THE INVENTION

The invention relates to films for the delivery of an active ingredient.

### BACKGROUND OF THE INVENTION

Film compositions that exhibit instant wettability followed by rapid dissolution/disintegration have been used to deliver or administer therapeutic or cosmetic substances, food flavor-imparting agents including food flavorings, or other ingredient contained within the film. Such films generally comprise a water-soluble edible polymer such as, for example, pullulan and/or starch. Upon exposure to an aqueous environment, e.g., the oral cavity, the film dissolves whereby the substance contained therein is released.

While numerous substances have been formulated in film form for oral delivery, some substances have proved difficult to incorporate into dissolvable films, or to incorporate at detectable levels, e.g., are not released into the mouth at levels high enough to impart a flavor burst or pharmacological effect. There is thus a need in the art for a method of preparing films that can be used for the delivery of such substances, in order to expand the types of substances that can be delivered using a film. The current invention addresses this need.

### SUMMARY OF THE INVENTION

The invention provides an active-containing dissolvable film prepared by forming a mixture comprising an active ingredient and film-forming ingredients, coating the mixture onto a substrate material to form a film, and then drying the film to a moisture content of less than about 15 weight % moisture, more typically less than about 10 weight % moisture. The active ingredient to be used in the practice of the invention is one having a water solubility of less than about 1 g/4 mL at room temperature. The active ingredient is present in the film in an amount sufficient to impart a desired action upon administration of a single dosage form of the active-containing dissolvable film. In one embodiment, the active is caffeine.

One embodiment of the invention provides a dissolvable caffeine-containing film comprising at least about 18% by dry weight of caffeine based on the weight of said film. Single dosage dissolvable films comprising from about 20 mg to about 30 mg of caffeine per single dosage film is a preferred embodiment.

The invention also provides a method of making an active-containing dissolvable film comprising an active ingredient. The method of the invention comprises forming a mixture of an active ingredient and film-forming ingredients, coating the mixture onto a substrate material to form a film, and then drying the film to a moisture content of less than about 15 weight % moisture, preferably less than about 10 weight % moisture. The active ingredient to be used in the practice of the invention is one having a water solubility of less than about 1 g/4 mL at room temperature. The active ingredient is used in amounts sufficient to impart a desired action upon administration of a single dosage form of the active-containing dissolvable film.

Also provided by the invention is a method of administering an active ingredient to an individual needing or desiring a particular active ingredient. The method comprises applying an active-containing dissolvable film to a moist area of an individual, upon which application the active is released. The active ingredient in the active-containing dissolvable film to be used in the practice of the invention is one having a water solubility of less than about 1 g/4 mL at room temperature. The active ingredient is present in the film in an amount sufficient to impart a desired action upon administration of a single dosage form of the active-containing dissolvable film. Ways in which the film may be administered include, but is not limited to, oral administration and topical administration.

### DETAILED DESCRIPTION OF THE INVENTION

The present invention relates to a film that can be used to administer a substance, referred to herein as an “active”, an “active ingredient”, an “active agent”, and the like, at levels sufficient or effective to impart a desired action. The terms “substance” and the term “active” are used interchangeably herein to refer to the ingredient intended for delivery, i.e., the film serves as the carrier or vehicle for delivery of the ingredient. Active ingredients include “drugs”, “bioactive agents,” “preparations,” “medicaments,” “therapeutic agents,” “physiological agents” and “pharmaceutical agents” and include substances for use in the diagnosis, cure, mitigation, arrest, treatment or prevention of a condition or disease state or to affect the structure or function of the body. Skin-wellness agents are included in this term.

By sufficient level or amount is meant that the active agent is present in amounts required to impart a desired action, such as a desired organoleptic, physiological or therapeutic effect. The active is present in an amount sufficient, also referred to herein as an effective amount, to bring about a desired result, e.g., a desired therapeutic result in the treatment of a condition. An effective amount of a drug, for example, means a nontoxic but sufficient amount of a drug to provide the selected effect over a specific period of time. The amount that constitutes a effective amount varies according to the particular active incorporated in the film, the condition being treated, any other actives being co-administered with the selected active, other components of the film, desired duration of treatment, the size of the film, and the like. Such an amount is readily determinable by the skilled practitioner.

The action exerted by the active may be perceived and measured in two ways. The level of active that is sufficient to impart a desired action can be measured by its perceived intensity and its perceived character. Intensity is defined as the overall strength of e.g., the taste, the smell or physiological reaction (e.g., strong, moderate, weak or slight, etc.). Character is defined as the perceived description of the of e.g., the taste, smell, physiological or pharmaceutical reaction (chocolate, peppermint, citrus, hightened alertness, amelioration of symptoms of disease or injury, and the like).

A single dosage form will typically be a single film, e.g., a strip of film formulated for oral delivery, but may refer to multiple films administered at substantially the same time. In the regard it is common in the medicinal art that one or two tablets are recommended, e.g., depending on body weight, age, or the like, for administration as a single dose.

The term “substantially aqueous environment” means the environment wherein the carrier film dissolves, more preferably rapidly dissolves, releasing the active. Typically, the substantially aqueous environment will be within the oral cavity, e.g., the surface on the tongue, or may be a food product such as a glass of water or juice, soup or the like. Encompassed are moist environments such as traumatized tissue resulting from a serious burn or the like.

The term rapidly dissolves means that the carrier film dissolves in less than about 60 seconds.

It is to be understood that both the film and active may dissolve in the aqueous environment or, alternatively, the film can dissolve and the active released into the aqueous environment, after which it may be swallowed or it may diffuse through the mucosal.

Preferred are actives that are water soluble at some level, and includes actives that can only be solubilized within a aqueous environment upon agitation, upon the application of heat, upon a change in pH, or upon application of heat and/or agitation and/or pH change. Preferred actives for use in the practice of the invention have a water solubility of less than about 1 g/4 mL at room temperature (22° C.). In one embodiment, the active is one having a solubility of less that about 1 g/10 mL at room temperature.

It has been discovered that some substances, when solubilized or dispersed in an aqueous environment can be incorporated into the film ingredients at levels sufficient to impart a desired action, such as a desired taste or a desired pharmacological or therapeutic effect.

One embodiment of the invention is directed to a dissolvable film comprising an active ingredient. In one embodiment, the active ingredients that are deliverable in accordance with the invention are actives that are water soluble at some level. A particularly preferred substance for incorporation into and delivery by means of a dissolvable film is caffeine.

In the practice of the invention, the active is solubilized or dispersed in an aqueous environment at or above room temperature. The active may be first solubilized or dispersed in water, and then the active-containing solution or suspension is mixed with the film forming ingredients to form a mixture. Alternatively, and more preferably, the active may be solubilized or dispersed in a solution of film forming ingredients to form a mixture. The mixture is then coated onto a suitable substrate to form a film and then dried to a moisture content of less than about 15 weight % moisture, more typically from about 5 weight % to about 15 weight % moisture, even more typically from about 6 weight % to about 10 weight % moisture. The formed film comprising the active substance can be air-dried or dried under warm air. The film may then be cut to the desired dimension, packaged and stored.

In one embodiment, the invention provides a dried water soluble film containing at least about 18% by dry weight of caffeine as the active substance. In a preferred embodiment, the prepared film comprises at least about 18% by dry weight, more preferably, at least about 20% by dry weight, and even more preferably at least about 25% by dry weight of caffeine, based on the weight of the finally formulated film.

In addition to the film forming ingredients and the desired active, the films of the invention may also comprise other ingredients such as flavor masking agents to cover-up the bitter or otherwise undesirable flavor of the chosen active.

Another embodiment of the invention is directed to a method of delivering a desired substance to a desired substrate, upon which delivery the desired substance is released. In the method of the invention the desired substance is solubilized or suspended in a dissolvable film, and the film is delivered to a desired substrate, said substrate comprising a substantially aqueous environment.

A further embodiment of the invention is directed to administering an active ingredient to an individual needing or desiring said active ingredient. The method comprises applying an active-containing dissolvable film to a moist area of an individual, such as the tongue or burned skin, upon which application the active is released. It will be appreciated that, in terms of compliance, the films of the invention are particularly useful in treating young children.

Treatment areas where the film of the invention finds use include treatment for antihistamines, pain management or anti-inflammatory, antiinflammatory conditions, incontinence, central nervous system conditions, hormone therapy and birth control, cardiovascular and cardiotonics, cosmetic, antinauseants, smoking cessation, both steroidal and nonsteroidal treatments, antibacterials, antiprotazoals, antifungals, calcium channel blockers, bronchodilators, enzyme inhibitors such as collagenase inhibitors, protease inhibitors, elastase inhibitors, lipoxygenase inhibitors, and angiotensin converting enzyme inhibitors, other antihypertensives, leukotriene antagonists, anti-ulceratives such as H2 antagonists, antivirals and/or immunomodulators, local anesthetics, antitussives, narcotic analgesics, cardioactive products such as atriopeptides, anticonvulsants, immunosuppressives, psychotherapeutics, sedatives, anticoagulants, analgesics, antimigrane agents, antiarrhythmic agents, antiemetics, anticancer agents, neurologic agents, hemostatics, anti-obesity agents, and the like.

Veterinary actives may also be administered using the films of the invention, as well as agricultural and horticultural agents. It will be appreciated that delivery in veterinary and horticultural applications enables more exact dosing, and less waste than administration in the food/irrigation water.

Specific examples of active agents include, but are not limited to benzocaine, caffeine, dextromethorphan hydrobromide, guaifenesin, loratidine, L-theanine, ompremazole, pseudoephedrine hydrochloride, and vitamins like niacin or retinol.

The film-forming composition used in the practice of the invention is not particularly limiting. The composition should be strong, flexible, be blocking and moisture resistant so that it does not adhere to itself or its packaging, yet be able to dissolve quickly when placed in a substantially aqueous environment.

Water soluble solid film-forming agents conventionally used in the dissolvable film-forming art can be used in the current invention. Such water soluble polymers include, but are not limited to pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and various mixtures thereof.

Pullulan is a natural neutral polysaccharide, repeatedly polymerized by maltotriose (three alpha 1,4 linkaged glucose) via alpha-1,6 linkages. It is a white powder that is tasteless, odorless, amorphous and non-crystalline. Pullulan is prepared by fermenting a starch hydrolyzate with the yeast Aureobasidium pullulan, filtering to remove cellular material, purifying, concentrating, drying and pulverizing. Transparent films can be made from an aqueous solution of pullulan.

Starch, as used herein, is intended to include all starches derived from any native source, any of which may be suitable for the films of the invention. A native starch, as this term is used herein, is one as it is found in nature. Also suitable are starches derived from a plant obtained by standard breeding techniques including crossbreeding, translocation, inversion, transformation or any other method of gene or chromosome engineering to include variations thereof. In addition, starch derived from a plant grown from artificial mutations and variations of the above generic composition, which may be produced by known standard methods of mutation breeding, are also suitable.

Typical sources for the starches include cereals, tubers, roots, legumes and fruits. The native source can be corn, pea, potato, sweet potato, banana, barley, wheat, rice, sago, amaranth, tapioca, arrowroot, canna, sorghum, and waxy or high amylose varieties thereof. A “waxy” starch is defined as a starch containing at least about 95% by weight amylopectin. A “high amylose” starch is defined as a starch containing at least about 40% by weight amylose.

Preferred starch-based films will comprise a modified starch. Preferably, at least about 50%, more preferably at least about 65%, and even more preferably at least about 90% of the starch will be a modified starch. The starch may be modified using any modification technique known in the art, including physical and/or chemical and/or enzymatic modifications.

Physically modified starches, such as sheared starches, or thermally-inhibited starches described in the family of patents represented by WO 95/04082, may be suitable for use herein.

Chemically modified products are also intended to be included as the base material and include, without limitation, those which have been crosslinked, acetylated and organically esterified, hydroxyethylated and hydroxypropylated, phosphorylated and inorganically esterified, cationic, anionic, nonionic, and zwitterionic, and succinate and substituted succinate derivatives thereof. Such modifications are known in the art, for example in Modified Starches: Properties and Uses, Ed. Wurzburg, CRC Press, Inc., Florida (1986).

Conversion products derived from any of the starches, including fluidity or thin-boiling starches prepared by oxidation, enzyme conversion, acid hydrolysis, heat and or acid clextrinization, thermal and or sheared products may also be useful herein.

Further suitable are pregelatinized starches which are known in the art and disclosed for example in U.S. Pat. Nos. 4,465,702, 5,037,929, 5,131,953, and 5,149,799. Conventional procedures for pregelatinizing starch are also known to those skilled in the art and described for example in Chapter XXII-“Production and Use of Pregelatinized Starch”, Starch: Chemistry and Technology, Vol. III, Industrial Aspects, R. L. Whistler and E. F. Paschall, Editors, Academic Press, New York 1967.

Any starch or starch blend having suitable properties, for use herein may be purified by any method known in the art to remove starch off flavors and colors that are native to the polysaccharide or created during processing. Suitable purification processes for treating starches are disclosed in the family of patents represented by EP 554 818 (Kasica, et al.). Alkali washing techniques, for starches intended for use in either granular or pregelatinized form, are also useful and described in the family of patents represented by U.S. Pat. No. 4,477,480 (Seidel) and U.S. Pat. No. 5,187,272 (Bertalan et al.).

Particularly suitable starches are starches capable of emulsifying or encapsulating the active ingredient so that there is no need for additional encapsulating or emulsifying agents, Such starches include, without limitation, hydroxyalkylated starches such as hydroxypropylated or hydroxyethylated starches, and succinated starches such as octenylsuccinated or dodecylsuccinated starches. The use of emulsifying or encapsulating starches are particularly useful in that a solution or dispersion of the film material (starch component, active agent, and optional additives) may be stored for later processing. The hydroxyalkylated starches have the added advantage of forming a softer film so that there is less or no need for a plasticizer.

The molecular weight of the starch is also important to its functionality in a film, particularly to film strength. For example, dextrins are not suitable in the present application.

The starch component may be a single modified starch, a blend of modified starches, or a blend of modified and native starches. Blends may be particularly useful to lower the cost of the film or to more easily achieve a variety of desirable properties and functionalities. If native starches are used, they may only be used in minor amounts, particularly less than 15%, more particularly less than about 10% by weight of the starch component.

The starch component may also comprise a cellulosic material or a gum, such as pullulan which is fully compatible and essentially substitutable for the starch. Other cellulosic materials and gums include without limitation carboxymethyl cellulose, hydroxypropyl cellulose, microcrystalline cellulose, ethylcellulose, cellulose acetate phthalate, hydrocolloids, carageenan, gums, and alginate. However, a cellulosic material or a gum is not an essential component of the film and may be used at levels of less than about 15 percent, more particularly less than about 10 percent by weight of the starch component, or may even be absent from the film. As starch is generally less expensive than pullulan, the cost of a pullulan film may be decreased by substituting starch for at least a portion of the pullulan, particularly at least about 50%, more particularly at least about 85%, most particularly at least about 90% of the pullulan by weight, without loss of the essential functionality of the pullulan film.

At least one plasticizer may be added to increase the apparent flexibility of the films. Further, a solid polyol plasticizer will generally provide better resistance to moisture absorption and blocking. One skilled in the art can chose a plasticizer to meet the desired needs of the film, such as chosing an edible plasticizer for an oral film. Plasticizers useful in the instant invention include, polyols, polycarboxylic acids, and polyesters. Examples of useful polyols include, but are not limited to ethylene glycol, propylene glycol, sugar alcohols such as sorbitol, manitol, maltitol, lactitol; mono-, di- and oligosaccharides such as fructose, glucose, sucrose, maltose, lactose, and high fructose corn syrup solids and ascorbic acid. Examples of polycarboxylic acids include, but are not limited to, citric acid, maleic acid, succinic acid, polyacrylic acid, and polymaleic acid. Examples of polyesters include but are not limited to glycerin triacetate, acetylated-monoglyceride, diethyl phthalate, triethyl citrate, tributyl citrate, acetyl triethyl citrate, acetyltributyl citrate. More typically, the plasticizer will be to glycerol, propylene glycol, sorbitol, and/or polyethylene glycol. The plasticizer may be present in any desired amount, particularly from 0 to about 50 percent, more particularly from 10 to about 30 by weight of the active containing formulated film.

Flavorings can be used as either a masking agent or as an active. Both natural and artificial flavors may be used. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnarnaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 26-dimethyloctanal (green fruit); and 2-dodecenal (citrus, mandarin); cherry, grape; mixtures thereof, and the like.

The amount of flavoring employed is normally a matter of preference subject to such factors as flavor type, individual flavor, strength desired and taste masking required. Thus, the amount may be varied in order to obtain the result desired in the final product. Such variations are within the capabilities of those skilled in the art without the need for undue experimentation. In general, amounts of about 0.1 to about 30 wt % are useable with amounts of about 2 to about 25 wt % being preferred and amounts from about 8 to about 15 wt % are more preferred.

Optional components may be added for a variety of reasons including without limitation, sweeteners, both natural and artificial; emulsifiers such as Polysorbate 80; humectants; surfactants; colorants, more particularly food grade colors; proteins such as gelatins; gums such as guar gum, and in addition to flavors, flavor enhancers. Such optional components are typically added in minor amounts, particularly less than about 30% total by weight based upon the weight of the final formulated product.

The film may be made by a variety of processes known in the art. For example, the starch may be dispersed with the other film components in water or other solvent and dried into film form. In the alternative, the starch and other dry components may be blended and then dispersed with any additional film components in water or other solvent and dried into film form. Films may be formed from such dispersions or solutions by shaping it into a solidified form of a suitable thickness by any technique known in the art including, but not limited to, wet casting, freeze-drying, and extrusion molding. The dispersion or solution may also be directly coated or sprayed onto another edible product, such as a tablet or foodstuff, and dried to form an edible film.

A particularly suitable process for preparing the films of the present invention is by preparing a coating formulation by making a solution of the film components, adding the active component, and applying heat to force the active into solution. The active is added in such amounts such that the final active-containing single dosage dissolvable film comprises a pre-determined effective amount. The target dosing level of caffeine, for example, will typically be from about 20 mg to about 30 mg of caffeine per strip, with a dosing of a single strip.

The prepared active and film forming containing mixture is applied to a substrate, using knife, bar or extrusion die coating methods, drying the coated substrate to remove the majority of the solvent, and removing the film from the substrate. Suitable substrates include, but are not limited to, silicone elastomers, metal foils and metalized polyfoils, composite foils or films containing polyetrafluoroethylene materials or equivalents thereof, polyether block amide copolymers, polyurethanes, polyvinylidene chloride, nylon, polyethylene, polyester, and other such materials useful in the art as releasable substrates.

The film is not completely dried in that some degree of water or other solvent remains. The amount of water may be controlled to obtain desired functionality. For example, more water typically results in a more flexible film, while too much water results in a film that will block (i.e., stacked films will adhere to one another and be difficult to separate) and be tacky. Typically, the films of the invention will have a moisture content of less than about 15 weight % moisture, preferably from about 5 weight % to about 15 weight % moisture, even more preferably from about 6 weight % to about 10 weight % moisture.

The film thickness will depend, in part, on the desired end use. Typically, the film thickness will be in the range of about 10 to 500 microns, particularly 25 to 200 microns. When prepared as an oral film for quick dissolution in the oral cavity, the film thickness is more preferably from about 50 to 150 microns. The films of the invention can be made in the form of an article such as a tape, a patch, a sheet, a dressing or any other form known to those skilled in the art. The dosage system may be produced in any desirable unit form. In addition to having various shapes, the dosage units produced may come in various sizes depending on the end use application (e.g., whether desinged for oral or topical administration).

Generally the device will be in the form of a strip of a size suitable to deliver a pre-selected amount of drug into the oral cavity without bending or folding the film. The thickness may vary over a wide range, typically from about 1 to about 5 mil, preferably from about 3 to about 5 mil thick, more typically from about 4 to about 5 mil thick. Generally a strip of about 1 inch in width, about 1¼ inch in length and about 4 mil in thickness (about 105 mg by weight) will be used for oral administration.

The films exhibit moisture and blocking resistance, yet are quickly wetted when exposed to water, such as when placed on the tongue or other substrate surface, followed by rapid dissolution. The wettability and dissolution rates of the starches may be modified by one skilled in the art to target a specific delivery profile. For example, more rapid dissolution is typically preferred when the film is an oral film while other uses, less rapid dissolution can be tolerated.

One skilled in the art can also modify the film formulation to provide clarity and other desired characteristics by manipulation of the pullulan and/or starch component and control of other components.

The films may be used for delivering any active agent for a variety of applications including personal care, skin care, wound care, pharmaceutical, and breath freshening. In addition to human applications, veterinary, agricultural and horticultural applications are contemplated.

The films of the invention may be stacked for multi-dose packaging or, if desired, be packaged in single dose form.

The following examples are presented for purpose of illustration only.

### EXAMPLE 1

40 g of modified food starch and 0.5 g carrageenan were dissolved in 100 g of 70° C. temperature using vigorous stirring. Once uniform, 10 g of glycerol and 5 g of propylene glycol, 12 g of flavor and 10 g of sweetener(s) were added with stirring. 20 g of caffeine was added to the uniform solution. A FD&C dye was then added to give the solution the desired color. Maintain mixing and temperature during the transfer of the solution from the mixing vessel to the coating station. The solution was coated and dried and subsequently converted into pieces suitable for oral consumption.

TABLE 1

| Film Ingredients | % |
|---|---|
| Modified starch | 20.0 |
| Caffeine | 10.0 |
| Mint Flavor | 6.0 |
| Sucralose | 5.0 |
| Glycerol | 5.0 |
| Propylene Glycol | 2.2 |
| Polysorbate 80 | 1.0 |
| Carrageenan | 0.25 |
| FD&C red color | 0.05 |
| Water | 50.5 |
| Total | 100.00 |

### EXAMPLE 2

40 g of modified food starch and 0.5 g gellan were dissolved in 10 g of 40° C. temperature using vigorous stirring. 10 g of glycerol and 5 g of propylene glycol, 10 g of flavor and 7 g of sweetener(s) were then added with stirring until the solution was uniform. 15 g of dextromethorphan hydrobromide was added to the uniform solution. A FD&C dye was then added to give the solution the desired color. The solution was coated and dried and subsequently converted into pieces suitable for oral consumption.

TABLE 2

| Film Ingredients | % |
|---|---|
| Modified starch | 20.0 |
| dextromethorphan hydrobromide | 7.5 |
| Cherry Flavor | 5.0 |
| Sucralose | 3.5 |
| Glycerol | 5.0 |
| Propylene Glycol | 1.7 |
| Polysorbate 80 | 1.0 |
| Gellan | 0.25 |
| FD&C red color | 0.05 |
| Water | 55.0 |
| Total | 100.00 |

Many modifications and variations of this invention can be made without departing from its spirit and scope, as will be apparent to those skilled in the art. The specific embodiments described herein are offered by way of examples only, and the invention is to be limited only by the terms of the appended claims, along with the full scope of equivalents to which such claims are entitled.

1. An active-containing dissolvable film prepared by forming a mixture comprising an active ingredient and film-forming ingredients, coating the mixture onto a substrate material to form a film, and then drying the film to a moisture content of less than about 15 weight % moisture, said active ingredient having a water solubility of less than about 1 g/4 mL at room temperature and being present in the film in amounts sufficient to impart a desired action upon administration of a single dosage form of the active-containing dissolvable film.

2. The film of claim 1 wherein the active ingredient has a water solubility of less than about 1 g/10 mL at room temperature.

3. The film of claim 1 wherein the mixture is in the form of a solution.

4. The film of claim 3 wherein a solution is formed upon the application of heat and/or agitation of the mixture.

5. The film of claim 1 wherein the mixture is in the form a suspension.

6. The film of claim 3 wherein said active ingredient is caffeine.

7. The film of claim 6 comprising at least about 18% by dry weight of caffeine based on the weight of the final formulated film.

8. The film of claim 7 comprising at least about 20% by dry weight of caffeine.

9. The film of claim 8 comprising at least about 25% by dry weight of caffeine.

10. The film of claim 1 wherein said film-forming ingredients comprise a starch component comprising at least about 85% modified starch.

11. The film of claim 10 wherein the starch is selected from the group consisting of a hydroxyalkylated starch and a succinated starch.

12. A method of making an active-containing dissolvable film comprising an active ingredient, the method comprising forming a mixture of an active ingredient and film-forming ingredients, coating the mixture onto a substrate material to form a film, and then drying the film to a moisture content of less than about 15 weight % moisture, said active ingredient having a water solubility of less than about 1 g/4 mL at room temperature and used in amounts sufficient to impart a desired action upon administration of a single dosage form of the active-containing dissolvable film.

13. The method of claim 12 wherein the mixture is in the form of a solution or in the form of a suspension.

14. A dissolvable caffeine-containing film comprising at least about 18% by dry weight of caffeine based on the weight of said film.

15. A single dosage dissolvable film of claim 14 comprising from about 20 mg to about 30 mg of caffeine per single dosage film.

16. A method of administering an active ingredient to an individual needing or desiring said active ingredient, said method comprising applying the active-containing dissolvable film of claim 1 to a moist area of an individual, upon which application the active is released.

17. The method of claim 16, wherein the film is applied to the tongue.

18. The method of claim 16, wherein the film is applied to traumatized tissue.

19. The method of claim 18 wherein the traumatized tissue is skin.

20. The method of claim 17 wherein the active ingredient is caffeine.
